(19) United States
(12) Patent Application Publication
(10) Pub. No.: US 2024/0115471 A1
(43) Pub. Date: Apr. 11, 2024

(54) NON-STICKY SMOOTH STABLE COMPOSITION

(71) Applicant: L'OREAL, Paris (FR)

(72) Inventors: Kazuhiko MARUYAMA, Kawasaki-shi (JP); Tomoko MIZUNO, Kawasaki-shi (JP)

(21) Appl. No.: 18/256,334

(22) PCT Filed: Dec. 3, 2021
(86) PCT No.: PCT/JP2021/045430

§ 371 Date: Jun. 7, 2023

(30) Foreign Application Priority Data
Dec. 11, 2020 (JP) 2020-205838
Jan. 13, 2021 (FR) 2100276

(51) Int. Cl. A61K 8/37; A61K 8/06; A61K 8/34; A61K 8/44; A61K 8/92; A61Q 19/00

(52) U.S. Cl. CPC A61K 8/375; A61K 8/062; A61K 8/342; A61K 8/442; A61K 8/927; A61Q 19/00; A61K 2800/10; A61K 2800/596

(57) ABSTRACT

The present invention relates to a composition, in the form of an O/W emulsion, comprising: (a) at least one oil; (b) at least one first polyglyceryl fatty acid ester having a C₆-C₂₂ fatty acid residue; (c) at least one second polyglyceryl fatty acid ester having a C₂₄-C₃₂ fatty acid residue; (d) at least one fatty alcohol; (e) at least one gemini surfactant; and (f) water, wherein the weighted average of HLB values of the (b) first polyglyceryl fatty acid ester and the (c) second polyglyceryl fatty acid ester is between 8 and 11, and the weight ratio of the amount of the (b) first polyglyceryl fatty acid ester/the amount of the (c) second polyglyceryl fatty acid ester is more than 15 and less than 35. The composition according to the present invention includes a polyglyceryl fatty acid ester, but it is stable and can provide no sticky feeling or a reduced sticky feeling as well as a non-greasy feeling or smooth feeling.

### TECHNICAL FIELD

The present invention relates to a composition, preferably a cosmetic or dermatological composition, which is non-sticky, smooth and stable.

### BACKGROUND ART

Compositions including polyglyceryl fatty acid esters have been known in the fields of cosmetics and dermatology. Polyglyceryl fatty acid esters can function as surfactants, and therefore, they may be used to prepare, typically, emulsions such as oil-in-water (O/W) or water-in-oil (W/O) emulsions.

For example, JP-A-2015-189762, JP-A-2014-073991, and JP-A-2017-114781 disclose emulsions which are formed by using a polyglyceryl fatty acid ester as a surfactant.

However, compositions including a polyglyceryl fatty acid ester often provide a sticky feeling to the touch or greasy feeling to the touch and tend to be unstable, in particular unstable over time and/or under temperature changes.

There is still a need for a new approach to reduce the sticky feeling to the touch of a composition comprising a polyglyceryl fatty acid ester and to make the composition stable.

### DISCLOSURE OF INVENTION

An objective of the present invention is to provide a composition which provides no sticky feeling or a reduced sticky feeling after application, as well as a non-greasy feeling or smooth feeling to the touch, and which is stable, in particular stable over time and/or under temperature changes, even if the composition includes a polyglyceryl fatty acid ester.

The above objective of the present invention can be achieved by a composition, in the form of an O/W composition, comprising:
(a) at least one oil;
(b) at least one first polyglyceryl fatty acid ester having a C₆-C₂₂ fatty acid residue;
(c) at least one second polyglyceryl fatty acid ester having a C₂₄-C₃₂ fatty acid residue;
(d) at least one fatty alcohol;
(e) at least one gemini surfactant; and
(f) water,
wherein
the weighted average of HLB values of the (b) first polyglyceryl fatty acid ester and the (c) second polyglyceryl fatty acid ester is between 8 and 11, and
the weight ratio of the amount of the (b) first polyglyceryl fatty acid ester/the amount of the (c) second polyglyceryl fatty acid ester is more than 15 and less than 35.

The (a) oil may be selected from the group consisting of ester oils, triglyceride oils and mixtures thereof.

The amount of the (a) oil in the composition according to the present invention may range from 0.01% to 15% by weight, preferably from 0.1% to 12% by weight, and more preferably from 1% to 9% by weight, relative to the total weight of the composition.

The (b) first polyglyceryl fatty acid ester may have a polyglyceryl moiety derived from 2 to 10 glycerins, preferably 2 to 8 glycerins, and more preferably from 2 to 6 glycerins.

The (b) first polyglyceryl fatty acid ester may be chosen from polyglyceryl monolaurate comprising 2 to 6 glycerol units, polyglyceryl monocaprate comprising 2 to 6 glycerol units, polyglyceryl monooleate comprising 2 to 6 glycerol units, and polyglyceryl distearate comprising 2 to 6 glycerol units.

The (b) first polyglyceryl fatty acid ester in the composition according to the present invention may range from 0.01% to 20% by weight, preferably from 0.05% to 15% by weight, and more preferably from 0.1% to 10% by weight, relative to the total weight of the composition.

The (c) second polyglyceryl fatty acid ester may have a polyglyceryl moiety derived from 2 to 10 glycerins, preferably 2 to 8 glycerins, and more preferably from 2 to 6 glycerins.

The (c) second polyglyceryl fatty acid ester may be polyglyceryl-3 beeswax.

The amount of the (c) second polyglyceryl fatty acid ester in the composition according to the present invention may range from 0.01% to 15% by weight, preferably from 0.05% to 10% by weight, and more preferably from 0.1% to 5% by weight, relative to the total weight of the composition.

The (d) fatty alcohol may be selected from the group consisting of cetyl alcohol, cetearyl alcohol, stearyl alcohol, behenyl alcohol, oleyl alcohol, and mixtures thereof.

The amount of the (d) fatty alcohol in the composition according to the present invention may range from 0.01% to 15% by weight, preferably from 0.05% to 10% by weight, and more preferably from 0.1% to 5% by weight, relative to the total weight of the composition.

The (e) gemini surfactant may be selected from the group consisting of sodium dilauramidoglutamide lysine, sodium dimyristoylglutamide lysine, sodium distearoylglutamide lysine, and a mixture thereof.

The amount of the (e) gemini surfactant in the composition according to the present invention may range from 0.01% to 5% by weight, preferably from 0.05% to 3% by weight, and more preferably from 0.1% to 1% by weight, relative to the total weight of the composition.

The amount of the (f) water in the composition according to the present invention may range from 50% to 90% by weight, preferably from 55% to 85% by weight, and more preferably from 60% to 80% by weight, relative to the total weight of the composition.

The present invention also relates to a cosmetic process for treating a keratin substance, comprising the step of applying the composition according to the present invention to the keratin substance.

### BEST MODE FOR CARRYING OUT THE INVENTION

After diligent research, the inventors have discovered a new approach to provide a composition which provides no sticky feeling or a reduced sticky feeling after application, as well as a non-greasy feeling or smooth feeling to the touch, and which is stable, in particular stable over time and/or under temperature changes, even if the composition includes a polyglyceryl fatty acid ester, by using a specific combination of selected ingredients under certain conditions.

Thus, one aspect of the present invention is a composition comprising:
(a) at least one oil;
(b) at least one first polyglyceryl fatty acid ester having a C₆-C₂₂ fatty acid residue;
(c) at least one second polyglyceryl fatty acid ester having a C₂₄-C₃₂ fatty acid residue;
(d) at least one fatty alcohol;
(e) at least one gemini surfactant; and
(f) water,
wherein
the weighted average of HLB values of the (b) first polyglyceryl fatty acid ester and the (c) second polyglyceryl fatty acid ester is between 8 and 11, and
the weight ratio of the amount of the (b) first polyglyceryl fatty acid ester/the amount of the (c) second polyglyceryl fatty acid ester is more than 15 and less than 35.

The composition according to the present invention includes a polyglyceryl fatty acid ester, but can provide no sticky feeling or a reduced sticky feeling as well as a non-greasy feeling or a smooth feeling to the touch.

Therefore, the composition according to the present invention can provide an excellent feeling during use, in particular on feeling of the skin during and after application of the composition.

The term “sticky” here means a property which provides a tacky feeling to the skin.

The term “greasy” here means a property which provides a slimy feeling to the skin.

The composition according to the present invention is stable just after the preparation of the composition and a long time after the preparation of the composition, even under temperature changes from cold to hot temperature. Therefore, the composition according to the present invention is stable over time, and can be stored for a long period of time even under temperature changes from winter to summer.

Hereinafter, the composition according to the present invention will be explained in a more detailed manner.

### [Oil]

The composition according to the present invention comprises (a) at least one oil. A single type of oil may be used, but two or more different types of oils may be used in combination.

Here, “oil” means a fatty compound or substance which is in the form of a liquid or a paste (non-solid) at room temperature (25° C.) under atmospheric pressure (760 mmHg). As the (a) oil(s), those generally used in cosmetics can be used alone or in combination thereof. These oils may be volatile or non-volatile.

The (a) oil may be a non-polar oil such as a hydrocarbon oil, a silicone oil, or the like; a polar oil such as a plant or animal oil and an ester oil or an ether oil; or a mixture thereof.

The (a) oil may be selected from the group consisting of oils of plant or animal origin, synthetic oils, silicone oils, and hydrocarbon oils.

As examples of plant oils, mention may be made of, for example, linseed oil, camellia oil, macadamia nut oil, corn oil, mink oil, olive oil, avocado oil, sasanqua oil, castor oil, safflower oil, jojoba oil, sunflower oil, almond oil, rapeseed oil, sesame oil, soybean oil, peanut oil, and mixtures thereof.

As examples of synthetic oils, mention may be made of alkane oils such as isododecane and isohexadecane, ester oils, ether oils, and artificial triglycerides.

The ester oils are preferably liquid esters of saturated or unsaturated, linear or branched C₁-C₂₆ aliphatic monoacids or polyacids and of saturated or unsaturated, linear or branched C₁-C₂₆ aliphatic monoalcohols or polyalcohols, the total number of carbon atoms of the esters being greater than or equal to 10.

Preferably, for the esters of monoalcohols, at least one from among the alcohol and the acid from which the esters of the present invention are derived is branched.

Among the monoesters of monoacids and of monoalcohols, mention may be made of ethyl palmitate, ethyl hexyl palmitate, isopropyl palmitate, dicaprylyl carbonate, alkyl myristates such as isopropyl myristate or ethyl myristate, isocetyl stearate, 2-ethylhexyl isononanoate, isononyl isononanoate, isodecyl neopentanoate and isostearyl neopentanoate.

Esters of C₄-C₂₂ dicarboxylic or tricarboxylic acids and of C₁-C₂₂ alcohols, and esters of monocarboxylic, dicarboxylic or tricarboxylic acids and of non-sugar C₄-C₂₆ dihydroxy, trihydroxy, tetrahydroxy or pentahydroxy alcohols may also be used.

Mention may especially be made of: diethyl sebacate; isopropyl lauroyl sarcosinate; diisopropyl sebacate; bis(2-ethylhexyl) sebacate; diisopropyl adipate; di-n-propyl adipate; dioctyl adipate; bis(2-ethylhexyl) adipate; diisostearyl adipate; bis(2-ethylhexyl) maleate; triisopropyl citrate; triisocetyl citrate; triisostearyl citrate; glyceryl trilactate; glyceryl trioctanoate; trioctyldodecyl citrate; trioleyl citrate; neopentyl glycol diheptanoate; diethylene glycol diisononanoate.

As ester oils, one can use sugar esters and diesters of C₆-C₃₀ and preferably C₁₂-C₂₂ fatty acids.

It is recalled that the term “sugar” means oxygen-bearing hydrocarbon-based compounds containing several alcohol functions, with or without aldehyde or ketone functions, and which comprise at least 4 carbon atoms. These sugars may be monosaccharides, oligosaccharides or polysaccharides.

Examples of suitable sugars that may be mentioned include sucrose (or saccharose), glucose, galactose, ribose, fucose, maltose, fructose, mannose, arabinose, xylose and lactose, and derivatives thereof, especially alkyl derivatives, such as methyl derivatives, for instance methylglucose.

The sugar esters of fatty acids may be chosen especially from the group comprising the esters or mixtures of esters of sugars described previously and of linear or branched, saturated or unsaturated C₆-C₃₀ and preferably C₁₂-C₂₂ fatty acids. If they are unsaturated, these compounds may have one to three conjugated or non-conjugated carbon-carbon double bonds.

The esters according to this variant may also be selected from monoesters, diesters, triesters, tetraesters and polyesters, and mixtures thereof.

These esters may be, for example, oleates, laurates, palmitates, myristates, behenates, cocoates, stearates, linoleates, linolenates, caprates and arachidonates, or mixtures thereof such as, especially, oleopalmitate, oleostearate and palmitostearate mixed esters, as well as pentaerythrityl tetraethyl hexanoate.

More particularly, use is made of monoesters and diesters and especially sucrose, glucose or methylglucose monooleates or dioleates, stearates, behenates, oleopalmitates, linoleates, linolenates and oleostearates.

An example that may be mentioned is the product sold under the name Glucate® DO by the company Amerchol, which is a methylglucose dioleate.

As examples of preferable ester oils, mention may be made of, for example, diisopropyl adipate, dioctyl adipate, 2-ethylhexyl hexanoate, ethyl laurate, cetyl octanoate, octyldodecyl octanoate, isodecyl neopentanoate, myristyl propionate, 2-ethylhexyl 2-ethylhexanoate, 2-ethylhexyl octanoate, 2-ethylhexyl caprylate/caprate, methyl palmitate, ethyl palmitate, isopropyl palmitate, dicaprylyl carbonate, isopropyl lauroyl sarcosinate, isononyl isononanoate, ethylhexyl palmitate, isohexyl laurate, hexyl laurate, isocetyl stearate, isopropyl isostearate, isopropyl myristate, isodecyl oleate, glyceryl tri(2-ethylhexanoate), pentaerythrithyl tetra(2-ethylhexanoate), 2-ethylhexyl succinate, diethyl sebacate, and mixtures thereof.

As examples of artificial triglycerides, mention may be made of, for example, capryl caprylyl glycerides, glyceryl trimyristate, glyceryl tripalmitate, glyceryl trilinolenate, glyceryl trilaurate, glyceryl tricaprate, glyceryl tricaprylate, glyceryl tri(caprate/caprylate) and glyceryl tri(caprate/caprylate/linolenate).

As examples of silicone oils, mention may be made of, for example, linear organopolysiloxanes such as dimethylpolysiloxane, methylphenylpolysiloxane, methylhydrogenpolysiloxane, and the like; cyclic organopolysiloxanes such as cyclohexasiloxane, octamethylcyclotetrasiloxane, decamethylcyclopentasiloxane, dodecamethylcyclohexasiloxane, and the like; and mixtures thereof.

Preferably, silicone oil is chosen from liquid polydialkylsiloxanes, especially liquid polydimethylsiloxanes (PDMS) and liquid polyorganosiloxanes comprising at least one aryl group.

These silicone oils may also be organomodified. The organomodified silicones that can be used in accordance with the present invention are silicone oils as defined above and comprise in their structure one or more organofunctional groups attached via a hydrocarbon-based group.

Organopolysiloxanes are defined in greater detail in Walter Noll's Chemistry and Technology of Silicones (1968), Academic Press. They may be volatile or non-volatile.

When they are volatile, the silicones are more particularly chosen from those having a boiling point of between 60° C. and 260° C., and even more particularly from:
(i) cyclic polydialkylsiloxanes comprising from 3 to 7 and preferably 4 to 5 silicon atoms. These are, for example, octamethylcyclotetrasiloxane sold in particular under the name Volatile Silicone® 7207 by Union Carbide or Silbione® 70045 V2 by Rhodia, decamethylcyclopentasiloxane sold under the name Volatile Silicone® 7158 by Union Carbide, Silbione® 70045 V5 by Rhodia, and dodecamethylcyclopentasiloxane sold under the name Silsoft 1217 by Momentive Performance Materials, and mixtures thereof. Mention may also be made of cyclocopolymers of the type such as dimethylsiloxane/methylalkylsiloxane, such as Silicone Volatile® FZ 3109 sold by the company Union Carbide, of formula:

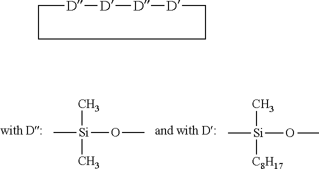

Mention may also be made of mixtures of cyclic polydialkylsiloxanes with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane;
(ii) linear volatile polydialkylsiloxanes containing 2 to 9 silicon atoms and having a viscosity of less than or equal to 5×10⁻⁶ m²/s at 25° C. An example is decamethyltetrasiloxane sold in particular under the name SH 200 by the company Toray Silicone. Silicones belonging to this category are also described in the article published in Cosmetics and Toiletries, Vol. 91, January 76, pp. 27-32, Todd & Byers, Volatile Silicone Fluids for Cosmetics. The viscosity of the silicones is measured at 25° C. according to ASTM standard 445 Appendix C.

Non-volatile polydialkylsiloxanes may also be used. These non-volatile silicones are more particularly chosen from polydialkylsiloxanes, among which mention may be made mainly of polydimethylsiloxanes containing trimethylsilyl end groups.

Among these polydialkylsiloxanes, mention may be made, in a non-limiting manner, of the following commercial products:
the Silbione® oils of the 47 and 70 047 series or the Mirasil® oils sold by Rhodia, for instance the oil 70 047 V 500 000;
the oils of the Mirasil® series sold by the company Rhodia;
the oils of the 200 series from the company Dow Corning, such as DC200 with a viscosity of 60 000 mm²/s;
the Viscasil® oils from General Electric and certain oils of the SF series (SF 96, SF 18) from General Electric.

Mention may also be made of polydimethylsiloxanes containing dimethylsilanol end groups known under the name dimethiconol (CTFA), such as the oils of the 48 series from the company Rhodia.

Among the silicones containing aryl groups are polydiarylsiloxanes, especially polydiphenylsiloxanes and polyalkylarylsiloxanes. Examples that may be mentioned include the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
certain oils of the SF series from General Electric, such as SF 1023, SF 1154, SF 1250 and SF 1265.

The organomodified liquid silicones may especially contain polyethyleneoxy and/or polypropyleneoxy groups. Mention may thus be made of the silicone KF-6017 proposed by Shin-Etsu, and the oils Silwet® L722 and L77 from the company Union Carbide.

Hydrocarbon oils may be chosen from:
linear or branched, optionally cyclic, C₆-C₁₆ lower alkanes. Examples that may be mentioned include hexane, undecane, dodecane, tridecane, and isoparaffins, for instance isohexadecane, isododecane and isodecane; and
linear or branched hydrocarbons containing more than 16 carbon atoms, such as liquid paraffins, liquid petroleum jelly, polydecenes and hydrogenated polyisobutenes such as Parleam®, and squalane.

As preferable examples of hydrocarbon oils, mention may be made of, for example, linear or branched hydrocarbons such as isohexadecane, isododecane, squalane, mineral oil (e.g., liquid paraffin), paraffin, vaseline or petrolatum, naphthalenes, and the like; hydrogenated polyisobutene, isoeicosan, and decene/butene copolymer; and mixtures thereof.

In one embodiment, the (a) oil may be chosen from triglyceride oils.

The triglyceride oil comprises at least one triglyceride. The triglyceride may be referred to as a triacyl glycerol, and three fatty acids or two fatty acids and one non-fatty acid, and one glycerol are esterified in the triglyceride.

The fatty acid may have, for example, 4 or more, 6 or more, 8 or more, or 10 or more carbon atoms, and 30 or fewer, 28 or fewer, 26 or fewer, or 24 or fewer carbon atoms. The fatty acid may have a different carbon chain length of, for example, from 4 to 30 carbon atoms, preferably from 6 to 28 carbon atoms, more preferably from 8 to 26 carbon atoms, and even more preferably from 10 to 24 carbon atoms. The carbon chain may be linear or branched.

The fatty acid may be saturated or unsaturated.

As examples of the saturated fatty acid, mention may be made of, for example, palmitic acid, heptadecanoic acid, stearic acid, nonadecanoic acid, arachidic acid, behenic acid, tetradocosanoic acid, hexadocosanoic acid, and octadocosanoic acid.

As examples of the unsaturated fatty acid, mention may be made of, for example, palmitoleic acid, oleic acid, elaidic acid, vaccenic acid, gadoleic acid, eicosenoic acid, erucic acid, nervonic acid, linoleic acid, eicosadienoic acid, docosadienoic acid, linolenic acid, pinolenic acid, eleostearic acid, mead acid, eicosatrienoic acid, stearidonic acid, arachidonic acid, eicosatetraenoic acid, adrenic acid, bosseopentaenoic acid, eicosapentaenoic acid, osbond acid, clupanodonic acid, tetracosapentaenoic acid, docosahexaenoic acid, and nisinic acid.

Examples of non-fatty acid may be dicarboxylic acids which may have, for example, 1 or more, 2 or more, 3 or more, or 4 or more carbon atoms, and 12 or fewer, 10 or fewer, 8 or fewer, or 6 or fewer carbon atoms. The non-fatty acid may have a different carbon chain length of, for example, from 1 to 12 carbon atoms, preferably from 2 to 10 carbon atoms, more preferably from 3 to 8 carbon atoms, and even more preferably from 4 to 6 carbon atoms. The carbon chain may be linear or branched.

The non-fatty acid, preferably dicarboxylic acid, may be saturated or unsaturated.

As examples of the saturated non-fatty acid, preferably saturated dicarboxylic acid, mention may be made of, for example, oxalic acid, malonic acid, succinic acid, glutaric acid, adipic acid, pimelic acid, suberic acid, azelaic acid and sebacic acid.

As examples of the unsaturated fatty acid, mention may be made of, for example, maleic acid, fumaric acid, citraconic acid, mesaconic acid and 2-pentenoic acid.

The triglyceride oil which may be suitable for the present invention is of plant origin. In other words, it is preferable that the triglyceride oil is selected from plant oils.

The plant oil may be selected from plant-extracted oils, plant-extracted butters, and mixtures thereof.

Among the plant-extracted oils, the following may be cited: jojoba oil, babassu oil, sunflower oil, olive oil, canola oil, coconut oil, meadowfoam seed oil; Brazil nut oil, marula oil, maize oil, argan oil, soybean oil, marrow oil, grapeseed oil, linseed oil, sesame oil, hazelnut oil, apricot oil, macadamia oil, arara oil, coriander oil, castor oil, avocado oil, shea butter oil, rapeseed oil, and copra oil.

Among the plant-extracted butters, the following may be cited: shea butter, Nilotica shea butter (Butyrospermum parkii), galam butter (Butyrospermum parkii), Borneo butter or fat or tengkawang tallow (Shorea stenoptera), shorea butter, illipe butter, madhuca butter or (Bassia) Madhuca longifolia butter, mowrah butter (Madhuca latifolia), katiau butter (Madhuca mottleyana), phulwara butter (M. butyracea), mango butter (Mangifera indica), murumuru butter (Astrocaryum murumuru), kokum butter (Garcinia indica), ucuuba butter (Virola sebifera), tucuma butter, painya (kpangnan) butter (Pentadesma butyracea), coffee butter (Coffea arabica), apricot butter (Prunus armeniaca), macadamia butter (Macadamia ternifolia), grapeseed butter (Vitis vinifera), avocado butter (Persea gratissima), olive butter (Olea europaea), sweet almond butter (Prunus amygdalus dulcis), cocoa butter (Theobroma cacao), and sunflower butter.

In a preferable embodiment, the triglyceride oil may be selected from caprylic/capric/succinic triglyceride, shea butter, and a mixture thereof.

It is preferable that the (a) oil be chosen from ester oils, triglyceride oils, hydrocarbon oils, silicone oils, and mixtures thereof, and more preferably chosen from ester oils, triglyceride oils and mixtures thereof.

It is preferable that the (a) oil be chosen from oils with a molecular weight below 600 g/mol.

The amount of the (a) oil(s) in the composition according to the present invention may be 0.01% by weight or more, preferably 0.1% by weight or more, and more preferably 1% by weight or more, relative to the total weight of the composition.

On the other hand, the amount of the (a) oil(s) in the composition according to the present invention may be 15% by weight or less, preferably 12% by weight or less, and more preferably 9% by weight or less, relative to the total weight of the composition.

The amount of the (a) oil(s) in the composition according to the present invention may range from 0.01% to 15% by weight, preferably from 0.1% to 12% by weight, more preferably from 1% to 9% by weight, relative to the total weight of the composition.

### [First Polyglyceryl Fatty Acid Ester]

The composition according to the present invention comprises (b) at least one first polyglyceryl fatty acid ester having a C₆-C₂ fatty acid residue. A single type of first polyglyceryl fatty acid ester may be used, but two or more different types of first polyglyceryl fatty acid ester may be used in combination.

The (b) first polyglyceryl fatty acid ester is a type of a nonionic surfactant.

It is preferable that the (b) first polyglyceryl fatty acid ester have a polyglycerol moiety derived from 2 to 10 glycerols, more preferably from 2 to 8 glycerols, and further more preferably from 2 to 6 glycerols.

The (b) first polyglyceryl fatty acid ester may have an HLB (Hydrophilic Lipophilic Balance) value of from 6.0 to 15.0, preferably from 6.5 to 14.5, and more preferably from 7.0 to 14.0.

The (b) first polyglyceryl fatty acid ester may be chosen from polyglyceryl fatty acid mono, di and tri esters. It is preferable that the (b) first polyglyceryl fatty acid ester be a polyglyceryl fatty acid monoester.

The (b) first polyglyceryl fatty acid ester has a C₆-C₂ fatty acid residue. The C₆-C₂₂ fatty acid residue may be derived from saturated or unsaturated fatty acids, preferably saturated fatty acids, including 6 to 22 carbon atoms, preferably 7 to 20 carbon atoms, and more preferably 8 to 18 carbon atoms, such as lauric acid, oleic acid, stearic acid, isostearic acid, capric acid, caprylic acid, and myristic acid.

The (b) first polyglyceryl fatty acid ester may be selected from the group consisting of PG2 caprylate, PG2 sesquicaprylate, PG2 dicaprylate, PG2 tricaprylate, PG2 caprate, PG2 sesquicaprate, PG2 dicaprate, PG2 tricaprate, PG2 laurate, PG2 sesquilaurate, PG2 dilaurate, PG2 trilaurate, PG2 myristate, PG2 sesquimyristate, PG2 dimyristate, PG2 trimyristate, PG2 stearate, PG2 sesquistearate, PG2 distearate, PG2 tristearate, PG2 isostearate, PG2 sesquiisostearate, PG2 diisostearate, PG2 triisostearate, PG2 oleate, PG2 sesquioleate, PG2 dioleate, PG2 trioleate, PG3 caprylate, PG3 sesquicaprylate, PG3 dicaprylate, PG3 tricaprylate, PG3 caprate, PG3 sesquicaprate, PG3 dicaprate, PG3 tricaprate, PG3 laurate, PG3 sesquilaurate, PG3 dilaurate, PG3 trilaurate, PG3 myristate, PG3 sesquimyristate, PG3 dimyristate, PG3 trimyristate, PG3 stearate, PG3 sesquistearate, PG3 distearate, PG3 tristearate, PG3 isostearate, PG3 sesquiisostearate, PG3 diisostearate, PG3 triisostearate, PG3 oleate, PG3 sesquioleate, PG3 dioleate, PG3 trioleate, PG4 caprylate, PG4 sesquicaprylate, PG4 dicaprylate, PG4 tricaprylate, PG4 caprate, PG4 sesquicaprate, PG4 dicaprate, PG4 tricaprate, PG4 laurate, PG4 sesquilaurate, PG4 dilaurate, PG4 trilaurate, PG4 myristate, PG4 sesquimyristate, PG4 dimyristate, PG4 trimyristate, PG4 stearate, PG4 sesquistearate, PG4 distearate, PG4 tristearate, PG4 isostearate, PG4 sesquiisostearate, PG4 diisostearate, PG4 triisostearate, PG4 oleate, PG4 sesquioleate, PG4 dioleate, PG4 trioleate, PG5 caprylate, PG5 sesquicaprylate, PG5 dicaprylate, PG5 tricaprylate, PG5 tetracaprylate, PG5 caprate, PG5 sesquicaprate, PG5 dicaprate, PG5 tricaprate, PG5 tetracaprate, PG5 laurate, PG5 sesquilaurate, PG5 dilaurate, PG5 trilaurate, PG5 tetralaurate, PG5 myristate, PG5 sesquimyristate, PG5 dimyristate, PG5 trimyristate, PG5 tetramyristate, PG5 stearate, PG5 sesquistearate, PG5 distearate, PG5 tristearate, PG5 tetrastearate, PG5 isostearate, PG5 sesquiisostearate, PG5 diisostearate, PG5 triisostearate, PG5 tetraisostearate, PG5 oleate, PG5 sesquioleate, PG5 dioleate, PG5 trioleate, PG5 tetraoleate, PG6 caprylate, PG6 sesquicaprylate, PG6 dicaprylate, PG6 tricaprylate, PG6 tetracaprylate, PG6 pentacaprylate, PG6 caprate, PG6 sesquicaprate, PG6 dicaprate, PG6 tricaprate, PG6 tetracaprate, PG6 pentacaprate, PG6 laurate, PG6 sesquilaurate, PG6 dilaurate, PG6 trilaurate, PG6 tetralaurate, PG6 pentalaurate, PG6 myristate, PG6 sesquimyristate, PG6 dimyristate, PG6 trimyristate, PG6 tetramyristate, PG6 pentamyristate, PG6 stearate, PG6 sesquistearate, PG6 distearate, PG6 tristearate, PG6 tetrastearate, PG6 pentastearate, PG6 isostearate, PG6 sesquiisostearate, PG6 diisostearate, PG6 triisostearate, PG6 tetraisostearate, PG6 pentaisostearate, PG6 oleate, PG6 sesquioleate, PG6 dioleate, PG6 trioleate, PG6 tetraoleate, PG6 pentaoleate, PG10 caprylate, PG10 sesquicaprylate, PG10 dicaprylate, PG10 tricaprylate, PG10 tetracaprylate, PG10 pentacaprylate, PG10 hexacaprylate, PG10 caprate, PG10 sesquicaprate, PG10 dicaprate, PG10 tricaprate, PG10 tetracaprate, PG10 pentacaprate, PG10 hexacaprate, PG10 laurate, PG10 sesquilaurate, PG10 dilaurate, PG10 trilaurate, PG10 tetralaurate, PG10 pentalaurate, PG10 hexalaurate, PG10 myristate, PG10 sesquimyristate, PG10 dimyristate, PG10 trimyristate, PG10 tetramyristate, PG10 pentamyristate, PG10 hexamyristate, PG10 stearate, PG10 sesquistearate, PG10 distearate, PG10 tristearate, PG10 tetrastearate, PG10 pentastearate, PG10 hexastearate, PG10 isostearate, PG10 sesquiisostearate, PG10 diisostearate, PG10 triisostearate, PG10 tetraisostearate, PG10 pentaisostearate, PG10 hexaisostearate, PG10 oleate, PG10 sesquioleate, PG10 dioleate, PG10 trioleate, PG10 tetraoleate, PG10 pentaoleate, and PG10 hexaoleate.

It is preferable that the (b) first polyglyceryl fatty acid ester be chosen from:
polyglyceryl monolaurate comprising 2 to 6 glycerol units,
polyglyceryl monocaprate comprising 2 to 6 glycerol units,
polyglyceryl monooleate comprising 2 to 6 glycerol units, and
polyglyceryl distearate comprising 2 to 6 glycerol units.

As examples of the (b) first polyglyceryl fatty acid ester, mention may be made of Sunsoft® Q-17D(G) from Taiyo Kagaku Co., Ltd., Sunsoft® A-121E-C from Taiyo Kagaku Co., Ltd., and Tegosoft® PC 41 from Evonik Co., Ltd.

The amount of the (b) first polyglyceryl fatty acid ester(s) in the composition according to the present invention may be 0.01% by weight or more, preferably 0.05% by weight or more, and more preferably 0.1% by weight or more, relative to the total weight of the composition.

On the other hand, the amount of the (b) first polyglyceryl fatty acid ester(s) in the composition according to the present invention may be 20% by weight or less, preferably 15% by weight or less, and more preferably 10% by weight or less, relative to the total weight of the composition.

The amount of the (b) first polyglyceryl fatty acid ester(s) in the composition according to the present invention may range from 0.01% to 20% by weight, preferably from 0.05% to 15% by weight, more preferably from 0.1% to 10% by weight, relative to the total weight of the composition.

### [Second Polyglyceryl Fatty Acid Ester]

The composition according to the present invention comprises (c) at least one second polyglyceryl fatty acid ester having C₂₄-C₃₂ fatty acid residue. A single type of second polyglyceryl fatty acid ester may be used, but two or more different types of second polyglyceryl fatty acid ester may be used in combination.

The (c) second polyglyceryl fatty acid ester is a type of a nonionic surfactant.

It is preferable that the (c) second polyglyceryl fatty acid ester have a polyglycerol moiety derived from 2 to 8 glycerols, more preferably from 2 to 6 glycerols, and further more preferably from 2 to 4 glycerols.

The (c) second polyglyceryl fatty acid ester may have an HLB (Hydrophilic Lipophilic Balance) value of from 3.0 to 7.0, preferably from 3.5 to 6.5, and more preferably from 4.0 to 6.0.

The (c) second polyglyceryl fatty acid ester may be chosen from polyglyceryl fatty acid mono, di and tri esters. It is preferable that the (c) second polyglyceryl fatty acid ester be a polyglyceryl fatty acid diester.

The (c) second polyglyceryl fatty acid ester has a C₂₄-C₃₂ fatty acid residue. The C₂₄-C₃₂ fatty acid residue may be derived from saturated or unsaturated fatty acids, preferably saturated fatty acids, including 24 to 32 carbon atoms, preferably 25 to 30 carbon atoms, and more preferably 26 to 28 carbon atoms, such as lignoceric acid, nervonic acid, cerotic acid, and montanic acid.

It is preferable that the (c) second polyglyceryl fatty acid ester having a C₂₄-C₃₂ fatty acid residue be a polyglyceryl diester represented by the following formula (B)

R³—C(O)—(O—CH₂—CH(OH)—CH₂)ₙ—O—C(O)—R⁴ (B)
wherein:
R³ and R⁴ denote, respectively, a linear or branched, saturated or unsaturated C₂₄ to C₃₂ fatty chain, and
n denotes 2 to 10, preferably 2 to 8 and more preferably 2 to 6.

According to an embodiment, in formula (B), the R³—C(O)— group corresponds to the carbon chain of a C₂₄ to C₃₂ fatty acid, said acid usually being linear and saturated, and preferably corresponds to a linear and saturated C₂₄ to C₃₀ fatty acid. This therefore includes, for example, tetracosanoic (or lignoceric) acid (C24) hexacosanoic (or cerotic) acid (C26). The R⁴ group corresponds to the hydrocarbon chain of an alcohol, said alcohol usually being saturated linear and having a C₂₄ to C₃₂ chain, preferably C₂₄ to C₃₀ chain.

According to the present invention, the polyglyceryl diester can be obtained by esterification of a solid wax in the presence of at least one polyol.

Depending on the source of the wax, the mixture of monoesters may also contain a certain proportion of hydroxyacid esters such as hydroxypalmitic or hydroxystearic acid. This is the case for example of beeswax. Preferably said alcohol is eicosanol, docosanol or tetracosanol. Beeswax, carnauba wax, candelilla wax, rice bran wax, sunflower wax, ouricury wax, Shellac wax and sugarcane wax are examples of natural solid waxes. Preferably, the solid wax is beeswax.

Solid waxes suitable for obtaining the polyglyceryl diester may have a melting point between 50 and 90° C. They correspond to mixtures essentially comprising monoesters having the formula R¹—C(O)—O—R², where the R¹—C(O)— group corresponds to the carbon chain of the fatty acid, said acid usually being linear and saturated and having a number of carbon atoms of at least 24, and in particular 26, and preferably up to 32 and preferably 30. This therefore includes hexacosanoic (or cerotic) acid (C26). Depending on the source of the wax, the mixture of monoesters may also contain a certain proportion of hydroxyacid esters such as hydroxypalmitic or hydroxystearic acid. This is the case for example of beeswax. The R² group corresponds to the hydrocarbon chain of an alcohol, said alcohol usually being saturated and linear and having a number of carbon atoms of at least 18, and in particular 20, and preferably up to 44 and preferably 34. Preferably said alcohol is eicosanol, docosanol or tetracosanol. Beeswax, carnauba wax, candelilla wax, rice bran wax, sunflower wax, ouricury wax, Shellac wax and sugarcane wax are examples of natural solid waxes.

Preferably, the solid wax suitable for the esterification reaction is beeswax.

Preferably, the polyol used for esterification is selected from the group comprising ethylene glycol, diethylene glycol, triethylene glycol, 2-methyl propanediol, propylene glycol, butylene glycol, neopentyl glycol, hexylene glycol, octylene glycol, polyethylene glycol, polypropylene glycol, trimethylol propane, sorbitol, erythritol, pentaerythritol, dipentaerythritol, glycerol, diglycerol and polyglycerol (i.e. a polymer of glycerol units). More preferably, the polyol is a polyglycerol, having an average degree of polymerization between 2 and 6, preferably of 3. Preferably, the polyol is polyglycerol-3.

The (c) second polyglyceryl fatty acid ester comprises the acid part of a solid wax. Waxes have a complex composition. They have the common feature of containing a mixture of acid monoesters and very long chain fatty alcohols.

Preferably, the (c) second polyglyceryl fatty acid ester is a wax derivative obtained by reacting together at least one solid wax in the presence of at least one polyol and optionally at least one catalyst. In such a case, a transesterification reaction occurs between the various chemical entities yielding the wax derivative.

The preferred catalysts are hydroxides or alkaline or alkaline earth alkoxides, calcium hydroxide, potassium or sodium carbonates or catalysts based on tin or titanium.

Preferably, the solid wax is advantageously selected from the group comprising carnauba wax, candelilla wax, rice bran wax, sunflower wax, sugarcane wax, ouricury wax, beeswax and Shellac wax.

In a preferred embodiment, the wax derivative is obtained by reacting beeswax and a polyglycerol such as polyglycerol-3.

In practice, the reaction is preferably conducted at a temperature of between 100° C. and 220° C., advantageously between 150° C. and 200° C.

It is preferable that the (c) second polyglyceryl fatty acid ester be polyglyceryl-3 beeswax.

The (c) second polyglyceryl fatty acid ester may be used as a combination with other ingredients.

For example, the (c) second polyglyceryl fatty acid ester may be used as a combination of jojoba wax, cetyl alcohol, polyglyceryl-6 distearate, and polyglyceryl-3 beeswax.

As the (c) second polyglyceryl fatty acid ester, EMULIUM® MELLIFERA (Gattefossé) comprising a mixture of jojoba wax, cetyl alcohol, polyglyceryl-6 distearate, and polyglyceryl-3 beeswax (INCI name: Polyglyceryl-6 Distearate (and) Jojoba Esters (and) Polyglyceryl-3 Beeswax (and) Cetyl Alcohol) may be used. This mixture comprises from 5% to 30% by weight of jojoba wax; from 3% to 15% by weight of cetyl alcohol; at least 50% by weight of polyglyceryl-6 distearate; and from 3% to 15% by weight of polyglyceryl-3 beeswax, relative to the total weight of the mixture.

The amount of the (c) second polyglyceryl fatty acid ester(s) in the composition according to the present invention may be 0.01% by weight or more, preferably 0.05% by weight or more, and more preferably 0.1% by weight or more, relative to the total weight of the composition.

On the other hand, the amount of the (c) second polyglyceryl fatty acid ester(s) in the composition according to the present invention may be 15% by weight or less, preferably 10% by weight or less, and more preferably 5% by weight or less, relative to the total weight of the composition.

The amount of the (c) second polyglyceryl fatty acid ester(s) in the composition according to the present invention may range from 0.01% to 15% by weight, preferably from 0.05% to 10% by weight, more preferably from 0.1% to 5% by weight, relative to the total weight of the composition.

### [Combination of First and Second Polyglyceryl Fatty Acid Esters]

According to the present invention, the weighted average of HLB values of the (b) first polyglyceryl fatty acid ester and the (c) second polyglyceryl fatty acid ester is between 8 and 11, preferably between 9 and 11, and more preferably between 9.5 and 10.5.

According to the present invention, the weight ratio of the amount of the (b) first polyglyceryl fatty acid ester/the amount of the (c) second polyglyceryl fatty acid ester is more than 15 and less than 35, preferably more than 17 and less than 30, and more preferably more than 19 and less than 25.

It is preferable that the total amount of the (b) first polyglyceryl fatty acid ester and the (c) second polyglyceryl fatty acid ester in the composition according to the present invention be 2% to 10% by weight, preferably 3% to 9% by weight, and more preferably 4% to 8% by weight, relative to the total weight of the composition.

### [Fatty Alcohol]

The composition according to the present invention comprises (d) at least one fatty alcohol. A single type of fatty alcohol may be used, but two or more different types of fatty alcohol may be used in combination.

The term “fatty” here means the inclusion of a relatively large number of carbon atoms. Thus, alcohols which have 6 or more, preferably 8 or more, and more preferably 10 or more carbon atoms are encompassed within the scope of fatty alcohols. The fatty alcohols may be saturated or unsaturated. The fatty alcohol may be linear or branched. Two or more fatty alcohols may be used in combination.

The (d) fatty alcohol may have the structure R—OH wherein R is chosen from saturated and unsaturated, linear and branched radicals containing from 8 to 40 carbon atoms, for example from 8 to 30 carbon atoms. In at least one embodiment, R is chosen from C₁₂-C₂₄ alkyl and C₁₂-C₂₄ alkenyl groups. R may be or may not be substituted with at least one hydroxyl group.

Non-limiting examples of the (d) fatty alcohols that may be mentioned include lauryl alcohol, cetyl alcohol, stearyl alcohol, oleyl alcohol, behenyl alcohol, linoleyl alcohol, undecylenyl alcohol, palmitoleyl alcohol, arachidonyl alcohol, erucyl alcohol, cetearyl alcohol, and a mixture thereof.

Examples of suitable fatty alcohols include, but are not limited to, cetyl alcohol, cetearyl alcohol, stearyl alcohol, behenyl alcohol, oleyl alcohol, and a mixture thereof.

The fatty alcohol may represent a mixture of fatty alcohols, which means that several species of fatty alcohol may coexist, in the form of a mixture, in a commercial product.

According to at least one embodiment, the fatty alcohol used in the composition according to the present invention is chosen from a mixture of cetyl alcohol and cetearyl alcohol (cetearyl alcohol).

The amount of the (d) fatty alcohol(s) in the composition according to the present invention may be 0.01% by weight or more, preferably 0.05% by weight or more, and more preferably 0.1% by weight or more, relative to the total weight of the composition.

On the other hand, the amount of the (d) fatty alcohol(s) in the composition according to the present invention may be 15% by weight or less, preferably 10% by weight or less, and more preferably 5% by weight or less, relative to the total weight of the composition.

The amount of the (d) fatty alcohol(s) in the composition according to the present invention may range from 0.01% to 15% by weight, preferably from 0.05% to 10% by weight, more preferably from 0.1% to 5% by weight, relative to the total weight of the composition.

### [Gemini Surfactant]

The composition according to the present invention comprises (e) at least one gemini surfactant. A single type of the gemini surfactant may be used, but two or more different types of the gemini surfactants may be used in combination.

The (e) gemini surfactant or dimeric surfactants used in the present invention are well known. For a detailed description of the various chemical structures and their physicochemical properties, reference may be made to the following publications:

Milton J. Rosen, Gemini Surfactants, Properties of surfactant molecules with two hydrophilic groups and two hydrophobic groups, Cosmetics & Toiletries magazine, Vol. 113, December 1998, pp. 49-55; and

Milton J. Rosen, Recent Developments in Gemini Surfactants, Allured's Cosmetics & Toiletries magazine, July 2001, Vol. 116, No. 7, pp. 67-70.

The (e) gemini surfactant may be selected from compounds with two or more fatty amide groups (hereafter, it may be referred to as “amide compound”). The amide compound may be represented by the following formula (A):

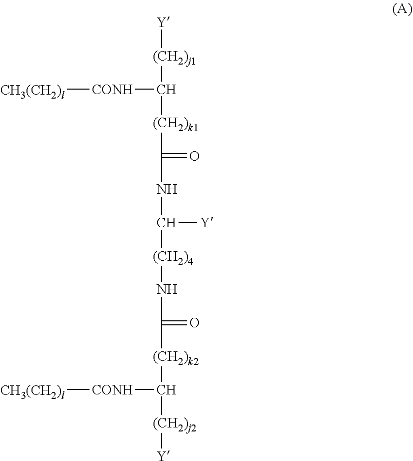

wherein
Y′ independently denotes a carboxylic acid group or an alkaline salt of a carboxylic acid group such as a sodium salt of a carboxylic acid group,
j1, k1, j2 and k2 denote an integer such that (j1, k1, j2, k2)=any of (2,0,2,0), (2,0,0,2), (0,2,2,0) and (0,2,0,2), and
l denotes an integer from 6 to 16, preferably 8 to 14, and more preferably 10 to 12.

Preferably, in formula (A), L denotes an integer from 8 to 12, j1=j2=0, and k1=k2=2.

Most preferably, in formula (A), Y′ is —CONa, j1=j2=0, k1=k2=2; and L=10.

The amide compound can be prepared by, for example, reacting a long chain N-acyl acidic amino acid anhydride with a basic amino acid, such as lysine, in water and/or a mixed solvent of water and organic solvent(s), or in inert organic solvent(s) such as tetrahydrofuran, benzene, toluene, xylene tetrachloromethane, chloroform, acetone or the like, or without any solvent, at −5° C. to 200° C., preferably 5° C. to 100° C., and more preferably 0° C. to 60° C.

As the amide compound, mention may be made of sodium dilauramidoglutamide lysine, sodium dimyristoylglutamide lysine, and sodium distearoylglutamide lysine.

Sodium dilauramidoglutamide lysine is in particular preferable. Sodium dilauramidoglutamide lysine is marketed as Pellicer L-10 and L-30 by Asahi Kasei Fine Chem. Col., Ltd. as an aqueous solution at a concentration of 29% by weight relative to the total weight of the aqueous solution; or as Pellicer LB-30G by Asahi Kasei Fine Chem. Co., Ltd. as a mixture of sodium dilauramidoglutamide lysine and of butyleneglycol.

In another embodiment, the (e) gemini surfactants can also comply with the following formula (I):

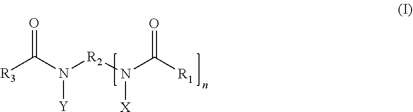

wherein:
R₁ and R₃ represent, independently of each other, an alkyl radical, comprising 1 to 25 carbon atoms;
R₂ represents a spacer group consisting of a linear or branched alkylene chain containing from 1 to 12 carbon atoms;
X and Y represent, independently of each other, a —(C₂H₄O)ᵣ—(C₃H₆O)_{b}Z group, wherein:
Z represents a hydrogen atom or the radical —CH₂—COOM, —SO₃M, —P(O)(OM)₂, —C₂H₄—SO₃M, —C₃H₆—SO₃M or —CH₂(CHOH)₄CH₂OH, wherein M represent H or an alkali metal or alkali earth metal ion or an ammonium or alkanolammonium ion;
a varies from 0 to 15, b varies from 0 to 10, and the sum of a+b varies from 1 to 25; and n varies from 1 to 10.

The gemini surfactant of formula (I) is preferably such that each one of the R₁—CO— and R₃—CO— groups comprises from 8 to 20 carbon atoms, and preferably represents a residue of coconut fatty acid (comprising mainly lauric acid and myristic acid).

In addition, this surfactant is preferably such that, for each one of the radicals X and Y, the sum of a and b has an average value that varies from 10 to 20 and is preferably equal to 15. A preferred group for Z is the —SO_{3M} group, where M is preferably an alkali metal ion such as the sodium ion.

The spacer R₂ consists advantageously of a C₁-C₃ linear alkylene chain and preferably an ethylene chain (CH₂CH₂). Finally, n is advantageously equal to 1.

A surfactant of this type is in particular the one identified by the INCI name: Sodium dicocoylethylenediamine PEG-15 sulfate, having the following structure:

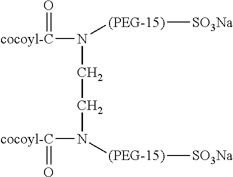

with the understanding that PEG represents the CH₂CH₂O group and that “cocoyl” represents the coconut fatty acid residue.

This surfactant has a molecular structure that is very similar to that of ceramide-3.

Preferably, the gemini surfactant according to the present invention may be used in a mixture with other surfactants, and in particular in a mixture with (a) an ester of a C₆-C₂₂ fatty acid (preferably C₁₄-C₂₀ such as stearate) and of glyceryl, (b) a diester of a C₆-C₂₂ fatty acid (preferably C₁₄-C₂₀ such as stearate) and of citric acid and glycerol (in particular a C₆-C₂₂ fatty acid ester and glyceryl monocitrate), and (c) a C₁₀-C₃₀ fatty alcohol (preferably behenyl alcohol).

Advantageously, the composition according to the present invention comprises a mixture of sodium dicocoylethylenediamine PEG-15 sulfate, of glyceryl stearate, of glyceryl stearate monocitrate and of behenyl alcohol.

For example, the gemini surfactant can be used as a mixture with other surfactants in the form of products sold by Sasol under the trade name Ceralution®, such as in particular the following products:
Ceralution® H: Behenyl Alcohol, Glyceryl Stearate, Glyceryl Stearate Citrate and Sodium Dicocoylethylenediamine PEG-15 Sulfate (INCI name),
Ceralution® F: Sodium Lauroyl Lactylate and Sodium Dicocoylethylenediamine PEG-15 Sulfate (INCI name), and
Ceralution® C: Aqua, Capric/Caprylic triglyceride, Glycerin, Ceteareth-25, Sodium Dicocoylethylenediamine PEG-15 Sulfate, Sodium Lauroyl Lactylate, Behenyl Alcohol, Glyceryl Stearate, Glyceryl Stearate Citrate, Gum Arabic, Xanthan Gum, Phenoxyethanol, Methylparaben, Ethylparaben, Butylparaben, Isobutylparaben (INCI name).

The gemini surfactant represents from 3% to 50% by weight of these mixtures. Preferably, the composition comprises, as a gemini surfactant, the compound with INCI name: Behenyl alcohol, glyceryl stearate, glyceryl stearate citrate and sodium dicocoylethylenediamine PEG-15 sulfate, sold under the trade name Ceralution® H by Sasol.

The amount of the (e) gemini surfactant(s) in the composition according to the present invention may be 0.01% by weight or more, preferably 0.05% by weight or more, and more preferably 0.1% by weight or more, relative to the total weight of the composition.

On the other hand, the amount of the (e) gemini surfactant(s) in the composition according to the present invention may be 5% by weight or less, preferably 3% by weight or less, and more preferably 1% by weight or less, relative to the total weight of the composition.

The amount of the (e) gemini surfactant(s) in the composition according to the present invention may range from 0.01% to 5% by weight, preferably from 0.05% to 3% by weight, and more preferably from 0.1% to 1% by weight, relative to the total weight of the composition.

### [Water]

The composition according to the present invention comprises (f) water.

The amount of (f) water in the composition according to the present invention may be 50% by weight or more, preferably 55% by weight or more, and more preferably 60% by weight or more, relative to the total weight of the composition.

On the other hand, the amount of (f) water in the composition according to the present invention may be 90% by weight or less, preferably 85% by weight or less, and more preferably 80% by weight or less, relative to the total weight of the composition.

The amount of (f) water in the composition according to the present invention may range from 50% to 90% by weight, preferably from 55% to 85% by weight, more preferably from 60% to 80% by weight, relative to the total weight of the composition.

### [Polyol]

The composition according to the present invention may further comprise at least one polyol. A single type of polyol may be used, but two or more different types of polyol may be used in combination.

The term “polyol” here means an alcohol having two or more hydroxy groups, and does not encompass a saccharide or a derivative thereof. The derivative of a saccharide includes a sugar alcohol which is obtained by reducing one or more carbonyl groups of a saccharide, as well as a saccharide or a sugar alcohol in which the hydrogen atom or atoms in one or more hydroxy groups thereof has or have been replaced with at least one substituent such as an alkyl group, a hydroxyalkyl group, an alkoxy group, an acyl group or a carbonyl group.

The polyol may be a C₂-C₁₂ polyol, preferably a C₂-C₉ polyol, comprising at least 2 hydroxy groups, and preferably 2 to 5 hydroxy groups.

The polyol may be a natural or synthetic polyol. The polyol may have a linear, branched or cyclic molecular structure.

The polyol may be selected from glycerins and derivatives thereof, and glycols and derivatives thereof.

The polyol may be selected from the group consisting of glycerin, diglycerin, polyglycerin, ethyleneglycol, diethyleneglycol, propyleneglycol, dipropyleneglycol, butyleneglycol, pentyleneglycol, hexyleneglycol, 1,3-propanediol, 1,5-pentanediol, polyethyleneglycol (5 to 50 ethyleneoxide groups), and sugars such as sorbitol.

The polyol may be present in an amount ranging from 0.01% to 30% by weight, and preferably from 0.1% to 20% by weight, such as from 1% to 10% by weight, relative to the total weight of the composition.

### [Other Optional Ingredients]

The composition according to the present invention may also comprise an effective amount of other optional ingredients, known previously elsewhere in cosmetic or dermatological compositions, such as various common adjuvants, hydrophilic or hydrophobic thickeners, sequestering agents such as EDTA and etidronic acid, preserving agents, vitamins or provitamins, for instance, panthenol, fragrances, plant extracts, cationic polymers and so on.

The composition according to the present invention may further comprise at least one organic solvent. So the organic solvent is preferably water miscible. As the organic solvent, there may be mentioned, for example, C₁-C₄ alkanols, such as ethanol and isopropanol; aromatic alcohols such as benzyl alcohol and phenoxyethanol; analogous products; and mixtures thereof.

The organic water-soluble solvents may be present in an amount ranging from less than 10% by weight, preferably from 5% by weight or less, and more preferably from 1% by weight or less, relative to the total weight of the composition.

### [Preparation and Properties]

The composition according to the present invention can be prepared by mixing the above essential and optional ingredients in accordance with a conventional process. The conventional process includes mixing with a homogenizer, for example a turbine mixer.

The composition according to the present invention is in the form of an O/W emulsion.

### [Process and Use]

It is preferable that the composition according to the present invention be a cosmetic composition, preferably a cosmetic composition for a keratin substance such as skin.

The composition according to the present invention can be used for a non-therapeutic process, such as a cosmetic process, for treating a keratin substance such as skin, hair, mucous membranes, nails, eyelashes, eyebrows and/or scalp, by being applied to the keratin substance. Thus, the present invention also relates to a cosmetic process for treating a keratin substance, comprising the step of applying the composition according to the present invention to the keratin substance.

The present invention may also relate to a use of the composition according to the present invention as a cosmetic product or in a cosmetic product such as care products, washing products, make-up products, make-up-removing products, for body and/or facial skin and/or mucous membranes and/or the scalp and/or the hair and/or the nails and/or the eyelashes and/or the eyebrows.

In other words, the composition according to the present invention can be used, as it is, as a cosmetic product. Alternatively, the composition according to the present invention can be used as an element of a cosmetic product. For example the composition according to the present invention can be added to or combined with any other elements to form a cosmetic product.

The care product may be a lotion, a cream, a hair tonic, a hair conditioner, a sun screening agent, and the like. The washing product may be a shampoo, a face wash, a hand wash and the like. The make-up product may be a foundation, a mascara, a lipstick, a lip gloss, a blusher, an eye shadow, a nail varnish, and the like. The make-up-removing product may be a make-up cleansing agent and the like.

### EXAMPLES

The present invention will be described in more detail by way of examples, which however should not be construed as limiting the scope of the present invention.

### Examples 1 and 2 and Comparative Examples 1-8

The following compositions according to Examples 1 and 2 and Comparative Examples 1-8, shown in Table 1, were prepared by mixing the components shown in Table 1 as follows:
(1) mixing the ingredients in column A in Table 1 at 70-75° C. to form a uniform mixture of Phase A;
(2) adding the ingredients in column B in Table 1 to the mixture of Phase A at 75-80° C., followed by homogenizing them to obtain a uniform mixture (Phases A and B) at 70-75° C.;
(3) mixing the ingredients in column C in Table 1 at 70-75° C. to form a mixture of Phase C;
(4) adding the mixture of Phase C to the mixture of Phases A and B at 70-75° C., followed by homogenizing them to obtain a uniform mixture (Phases A, B and C) at 70-75° C. and cooling the obtained mixture to about 30° C.; and
(5) adding the ingredient in columns D and E in Table 1 to the mixture of Phases A, B and C at about 30° C., followed by homogenizing them to obtain a uniform mixture (Phases A, B, C, D and E) at about 30° C., and cooling the obtained mixture to room temperature.

The numerical values for the amounts of the components shown in Table 1 are all based on “% by weight” as active materials.

TABLE 1

| | | | | | Comp. | Comp. | Comp. | Comp. | Comp. | Comp. | Comp. | Comp. |
|---|---|---|---|---|---|---|---|---|---|---|---|---|
| | | Ingredients | Ex. 1 | Ex. 2 | Ex. 1 | Ex. 2 | Ex. 3 | Ex. 4 | Ex. 5 | Ex. 6 | Ex. 7 | Ex. 8 |
| B | b) | Polyglyceryl-5 Laurate (HLB: 10.9) | 3.5 | — | — | — | — | — | 3.5 | 3.5 | 3.5 | 3.5 |
| | | Polyglyceryl-2 Oleate (HLB: 7) | — | 1.7 | — | 3.5 | 3.5 | — | — | — | — | — |
| | | Polyglyceryl-4 Caprate (HLB: 14) | — | 1.8 | — | — | — | 3.5 | — | — | — | — |
| | | Polyglyceryl-6 Distearate (HLB: 9) | 1.92 | 1.92 | 1.92 | 4.48 | 1.92 | 1.92 | 1.92 | 1.92 | 1.92 | 1.92 |
| | c) | Polyglyceryl-3 Beeswax (HLB: 4.5) | 0.255 | 0.255 | 0.255 | 0.595 | 0.255 | 0.255 | — | 0.255 | 0.255 | 0.255 |
| | | Jojoba Esters | 0.57 | 0.57 | 0.57 | 1.33 | 0.57 | 0.57 | 0.57 | 0.57 | 0.57 | 0.57 |
| | d) | Cetyl Alcohol | 0.255 | 0.255 | 0.255 | 0.595 | 0.255 | 0.255 | 0.255 | 0.255 | — | 0.255 |
| | a) | Isopropyl Lauroyl Sarcosinate | 5 | 5 | 5 | 5 | 5 | 5 | 5 | — | 5 | 5 |
| | | Isopropyl Myristate | 1.3 | 1.3 | 1.3 | 1.3 | 1.3 | 1.3 | 1.3 | — | 1.3 | 1.3 |
| | | Butyrospermum Parkii (Shea) Butter | 0.9 | 0.9 | 0.9 | 0.9 | 0.9 | 0.9 | 0.9 | — | 0.9 | 0.9 |
| | | Diisopropyl Adipate | 0.9 | 0.9 | 0.9 | 0.9 | 0.9 | 0.9 | 0.9 | — | 0.9 | 0.9 |
| | d) | Bchenyl Alcohol | 1.5 | 1.5 | 1.5 | 1.5 | 1.5 | 1.5 | 1.5 | 1.5 | — | 1.5 |
| A | e) | Sodium Dilauramidoglutamide Lysine | 0.35 | 0.35 | 0.35 | 0.35 | 0.35 | 0.35 | 0.35 | 0.35 | 0.35 | — |
| | f) | Water | qsp100 | qsp100 | qsp100 | qsp100 | gsp100 | qsp100 | qsp100 | qsp100 | qsp100 | qsp100 |
| | | Glycerin | 10 | 10 | 10 | 10 | 10 | 10 | 10 | 10 | 10 | 10 |
| | | Chlorphenesin | 0.2 | 0.2 | 0.2 | 0.2 | 0.2 | 0.2 | 0.2 | 0.2 | 0.2 | 0.2 |
| | | Adenosine | 0.1 | 0.1 | 0.1 | 0.1 | 0.1 | 0.1 | 0.1 | 0.1 | 0.1 | 0.1 |
| | | Hydroxyacetophenone | 0.2 | 0.2 | 0.2 | 0.2 | 0.2 | 0.2 | 0.2 | 0.2 | 0.2 | 0.2 |
| | | Caprylyl Glycol | 0.2 | 0.2 | 0.2 | 0.2 | 0.2 | 0.2 | 0.2 | 0.2 | 0.2 | 0.2 |
| | | Tetrasodium Glutamate Diacetate | 0.2 | 0.2 | 0.2 | 0.2 | 0.2 | 0.2 | 0.2 | 0.2 | 0.2 | 0.2 |
| | | Citric Acid | 0.04 | 0.04 | 0.04 | 0.04 | 0.04 | 0.04 | 0.04 | 0.04 | 0.04 | 0.04 |
| | | Sodium Stearoyl Glutamate | 0.1 | 0.1 | 0.1 | 0.1 | 0.1 | 0.1 | 0.1 | 0.1 | 0.1 | 0.1 |
| B | | Pentaerythrityl Tetra-di-t-butyl | 0.07 | 0.07 | 0.07 | 0.07 | 0.07 | 0.07 | 0.07 | 0.07 | 0.07 | 0.07 |
| | | Hydroxyhydrocinnamate | | | | | | | | | | |
| | | Capryloyl Salicylic Acid | 0.3 | 0.3 | 0.3 | 0.3 | 0.3 | 0.3 | 0.3 | 0.3 | 0.3 | 0.3 |
| C | | Ammonium Polyacryloyldimethjyl | 0.5 | 0.5 | 0.5 | 0.5 | 0.5 | 0.5 | 0.5 | 0.5 | 0.5 | 0.5 |
| | | Taurate | | | | | | | | | | |
| | | Xanthan Gum (and) Ceratonia Siliqua | 0.1 | 0.1 | 0.1 | 0.1 | 0.1 | 0.1 | 0.1 | 0.1 | 0.1 | 0.1 |
| | | (Carob) Gum | | | | | | | | | | |
| D | | Bisaccharide Gum-2 | 5 | 5 | 5 | 5 | 5 | 5 | 5 | 5 | 5 | 5 |
| E | | Ethanol | 3 | 3 | 3 | 3 | 3 | 3 | 3 | 3 | 3 | 3 |

| Total Amount (wt %) | 100 | 100 | 100 | 100 | 100 | 100 | 100 | 100 | 100 | 100 |
|---|---|---|---|---|---|---|---|---|---|---|
| Average HLB (PGFEs) | 10.0 | 9.8 | 8.5 | 7.9 | 7.6 | 11.9 | 10.2 | 10.0 | 10.0 | 10.0 |
| b)/c) | 21.3 | 21.3 | 7.5 | 13.4 | 21.3 | 21.3 | 0 | 21.3 | 21.3 | 21.3 |
| Non-Sticky Feeling | Good | Very | Good | Good | Good | Good | Poor | Very | Poor | Poor |
| | | Good | | | | | | Poor | | |
| Smooth Feeling (Non-Greasy Feeling) | Very | Very | Good | Good | Good | Good | Poor | Very | Poor | Good |
| | Good | Good | | | | | | Poor | | |
| Temperature Stability | Very | Very | Poor | Poor | Poor | Poor | Good | Very | Good | Poor |
| | Good | Good | | | | | | Poor | | |

### [Evaluations]

### (Non-Sticky Feeling)

5 professional panelists evaluated “non-sticky feeling” after the application for the compositions according to Examples 1 and 2 and Comparative Examples 1-8. Each panelist took each composition in their hands, then applied it onto their faces to evaluate non-sticky feeling after application, and graded it from 1 (poor) to 5 (very good), which was then classified in the following 4 categories based on the average of the grade:
Very Good: From 5.0 to 4.0
Good: From 3.9 to 3.0
Poor: From 2.9 to 2.0
Very Poor: From 1.9 to 1.0

The results are shown in Table 1.

### (Smooth Feeling)

5 professional panelists evaluated “smooth feeling (non-greasy feeling)” during application for the compositions according to Examples 1 and 2 and Comparative Examples 1-8. Each panelist took each composition in their hands, then applied it onto their faces to evaluate smooth feeling (non-greasy feeling), and graded it from 1 (poor) to 5 (very good), which was then classified in the following 4 categories based on the average of the grade:
Very Good: From 5.0 to 4.0
Good: From 3.9 to 3.0
Poor: From 2.9 to 2.0
Very Poor: From 1.9 to 1.0

The results are shown in Table 1.

### (Temperature Stability)

Each of the compositions according to Examples 1 and 2 and Comparative Examples 1-8 was filled into a glass bottle and was held under temperature changing conditions at 4° C., 25° C., 40° C. and 45° C. for 2 months. Each sample was then investigated for the degree of change (color, odor, and pH), and evaluated in accordance with the following criteria:
Very Good: Almost the same conditions as at production.
Good: Changes in color, odor, and pH were somewhat observed. However, no separation or no creaming or crystallizing aspect was observed.
Poor: Changes in color, odor, and pH were clearly observed. Separation, or creaming or crystallizing aspect was clearly observed.
Very Poor: Changes in color, odor, and pH were remarkably noticed. Separation, or creaming or crystallizing aspect was remarkably noticed.

The results are shown in Table 1.

As is clear from Table 1, the compositions in the form of an O/W emulsion according to the present invention (Examples 1 and 2) were able to provide excellent cosmetic effects in terms of non-sticky feeling, smooth feeling, and stability under temperature changes for 2 months, which could be attributed to a combination of ingredients (b) and (c) under certain conditions. Thus, the composition according to the present invention can provide, in particular, an excellent feeling to the touch and stability for a long time period even under temperature changes.

On the other hand, Comparative Example 1 which does not satisfy the condition that the weight ratio of the amount of ingredient (b)/the amount of ingredient (c) is more than 15 and less than 35, showed inferior temperature stability.

Comparative Example 2 which does not satisfy the conditions of (i) the weighted average of HLB values of ingredients (b) and (c) is between 8 and 11, and (ii) the weight ratio of the amount of ingredient (b)/the amount of ingredient (c) is more than 15 and less than 35, showed inferior temperature stability. In particular, in Comparative Example 2, crystallization was observed in the stability evaluation.

Comparative Examples 3 and 4 which do not satisfy the condition that the weighted average of HLB values of ingredients (b) and (c) is between 8 and 11, showed inferior temperature stability.

Comparative Example 5 which does not use ingredient (c) showed inferior non-sticky feeling or inferior smooth feeling.

Comparative Example 6 which does not use ingredient (a) showed very poor cosmetic effects in terms of any of non-sticky feeling, smooth feeling and temperature stability.

Comparative Example 7 which does not use ingredient (d) showed inferior non-sticky feeling or inferior smooth feeling.

Comparative Example 8 which does not use ingredient (e) showed inferior non-sticky feeling or inferior temperature stability.

1. A composition, in the form of an O/W emulsion, comprising:
(a) at least one oil;
(b) at least one first polyglyceryl fatty acid ester having a C₆-C₂₂ fatty acid residue;
(c) at least one second polyglyceryl fatty acid ester having a C₂₄-C₃₂ fatty acid residue;
(d) at least one fatty alcohol;
(e) at least one gemini surfactant; and
(f) water,
wherein
the weighted average of HLB values of the (b) first polyglyceryl fatty acid ester and the (c) second polyglyceryl fatty acid ester is between 8 and 11, and
the weight ratio of the amount of the (b) first polyglyceryl fatty acid ester/the amount of the (c) second polyglyceryl fatty acid ester is more than 15 and less than 35.

2. The composition according to claim 1, wherein the (a) oil is selected from the group consisting of ester oils, triglyceride oils and mixtures thereof.

3. The composition according to claim 1, wherein the amount of the (a) oil in the composition ranges from 0.01% to 15% by weight, relative to the total weight of the composition.

4. The composition according to claim 1, wherein the (b) first polyglyceryl fatty acid ester has a polyglyceryl moiety derived from 2 to 10 glycerins.

5. The composition according to claim 1, wherein the (b) first polyglyceryl fatty acid ester is chosen from polyglyceryl monolaurate comprising 2 to 6 glycerol units, polyglyceryl monocaprate comprising 2 to 6 glycerol units, polyglyceryl monooleate comprising 2 to 6 glycerol units, and polyglyceryl distearate comprising 2 to 6 glycerol units.

6. The composition according to claim 1, wherein the amount of the (b) first polyglyceryl fatty acid ester in the composition ranges from 0.01% to 20% by weight, relative to the total weight of the composition.

7. The composition according to claim 1, wherein the (c) second polyglyceryl fatty acid ester has a polyglyceryl moiety derived from 2 to 10 glycerins.

8. The composition according to claim 1, wherein the (c) second polyglyceryl fatty acid ester is polyglyceryl-3 beeswax.

9. The composition according to claim 1, wherein the amount of the (c) second polyglyceryl fatty acid ester in the composition ranges from 0.01% to 15% by weight, relative to the total weight of the composition.

10. The composition according to claim 1 wherein the (d) fatty alcohol is selected from the group consisting of cetyl alcohol, cetearyl alcohol, stearyl alcohol, behenyl alcohol, oleyl alcohol, and mixtures thereof.

11. The composition according to claim 1, wherein the amount of the (d) fatty alcohol in the composition ranges from 0.01% to 15% by weight, relative to the total weight of the composition.

12. The composition according to claim 1 wherein the (e) gemini surfactant is selected from the group consisting of sodium dilauramidoglutamide lysine, sodium dimyristoylglutamide lysine, sodium distearoylglutamide lysine, and a mixture thereof.

13. The composition according to claim 1, wherein the amount of the (e) gemini surfactant in the composition ranges from 0.01% to 5% by weight, relative to the total weight of the composition.

14. The composition according to claim 1, wherein the amount of the (f) water in the composition ranges from 50% to 90% by weight, relative to the total weight of the composition.

15. A cosmetic process for treating a keratin substance, comprising applying the composition according to claim 1 to the keratin substance.
